(19) United States
(12) Patent Application Publication
(10) Pub. No.: US 2008/0253978 A1
(43) Pub. Date: Oct. 16, 2008

(54) Derivatives of Pyrrolyltriazine Together with Methods for Obtaining Them and Their Use as Protecting Agents Uv Radiation

(75) Inventors: Carles Trullas, Barcelona (ES); Carles Pelejero, Barcelona (ES); David Panyella, Barcelona (ES); Jordi Corbera, Barcelona (ES); Jorg Holenz, Barcelona (ES); David Vano, Barcelona (ES)

Correspondence Address: BERENBAUM, WEINSHIENK & EASON, P.C, 370 17TH STREET, SUITE 4800, DENVER, CO, 80202, US

(73) Assignee: ISDIN, S.A., Barcelona (ES)

(21) Appl. No.: 11/916,450

(22) PCT Filed: Jun. 6, 2006
(86) PCT No.: PCT/EP06/62937

§ 371 Date: Dec. 3, 2007

(30) Foreign Application Priority Data
Jun. 3, 2005 (ES) P-200501334

(51) Int. Cl. A61K 8/49; C07D 417/04; A61Q 19/00

(52) U.S. Cl. 424/59; 544/185

(57) ABSTRACT

The present invention relates to new pyrrolyltriazine derivatives of general formula (I) together with method for obtaining them. The physico-chemical properties of said compounds allow them to be used as absorbents of UV radiation.

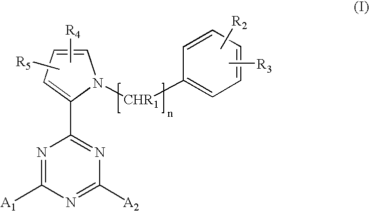

### FIELD OF THE INVENTION

The present invention is related to the cosmetic, dermatological and pharmaceutical fields. In particular, the present invention relates to new derivatives of pyrrolyltriazine which, due to their physico-chemical properties, are useful as protecting agents against UV radiation, together with their use for manufacturing cosmetic, dermatological and pharmaceutical formulations that protect the skin, lips, nails and hair against UV radiation.

### BACKGROUND OF THE INVENTION

Sunlight, and ultraviolet radiation in particular, can under certain circumstances provoke harmful effects on the skin, giving rise to pathological manifestations such as burns, photodermatosis and photoageing, among others.

The main factor responsible for such pathological manifestations is ultraviolet radiation, whose energy is inversely proportional to its wavelength. Thus, the shorter the wavelength the more energetic the radiation is. Ultraviolet radiation can be classified into UV-C, UV-B and UV-A, with UV-C being the most harmful, although it is absorbed by the ozone layer.

To counteract the damage that UV radiation-A and UV-B can cause, people's skin has various natural protection systems that either absorb or deflect the radiation, such as melanin, hair, the fatty layer of the skin, etc.

Solar filters are currently used in this respect in order to reduce the effects of solar radiation. Such solar filters are compounds that are applied to the skin, lips, nails or hair and that can be found included in cosmetic, dermatological and pharmaceutical formulations and in other cosmetic preparations to protect against solar radiation, preventing the decomposition of active substances or components sensitive to radiation.

Research has been carried out in recent years into compounds whose physico-chemical properties would be more effective as solar filters or sunscreens.

An example would be found in the document WO 03/075875, which discloses compositions that absorb UV radiation including a hydroxyphenyltriazine compound of general formula (1):

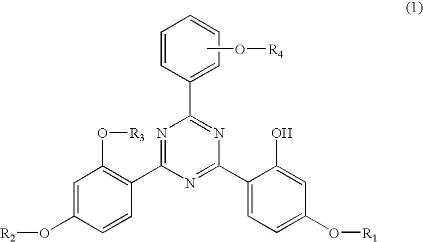

in which R₁, R₂ and R₃ are independently C₁₋₁₈ alkyl, C₂₋₁₀ alkenyl or C₁₋₄ phenylalkyl, and R₄ is hydrogen or C₁-C₅ alkyl.

Despite the wide diversity of solar filters or sunscreens, there exists a need for new compounds whose physico-chemical properties make them suitable solar filters for simultaneous protection against UV-A and UV-B radiation.

### DESCRIPTION OF THE INVENTION

A first aspect of the present invention is a pyrrolyltriazine derivative of general formula (I):

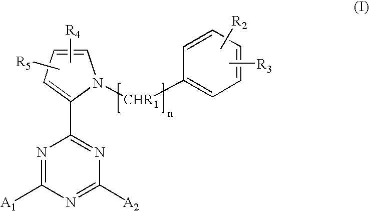

in which
n=0, 1, 2, 3 or 4;

R₁ represents an atom of hydrogen; linear or branched chain alkyl radical having from 1 to 3 atoms, optionally substituted; or a substituted R₂′, R₃′ phenyl radical;

R₂, R₂′, R₃ and R₃′ are the same as or different from each other and represent a hydrogen; an optionally substituted linear or branched chain alkyl radical having from 1 to 3 carbon atoms; an alkoxy radical having from 1 to 3 carbon atoms; an aryl radical; halogen or hydroxyl with the condition that when n=0, R₂ or R₃ are not an acryl derivative; or

R₂ and R₃ form with the phenyl a naphthalene ring, optionally substituted;

R₄ and R₅ are same as or different from each other and represent an atom of hydrogen; an optionally substituted linear or branched chain alkyl radical having from 1 to 4 carbon atoms; or an optionally substituted aryl radical;

A₁ is a radical of general formula (II), (III) or (IV)

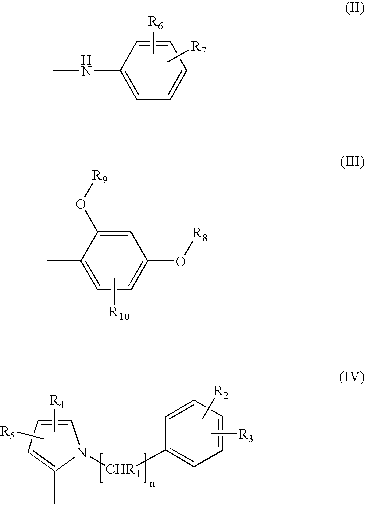

A₂ is a radical of general formula (II) or (V)

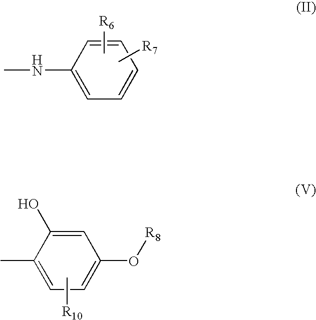

R₆ represents an atom of hydrogen; a linear or branched chain, saturated or unsaturated alkyl radical optionally substituted that contains from 1 to 6 carbon atoms; or a hydroxyl radical;

R₇ represents an atom of hydrogen; an optionally substituted aryl radical; an optionally substituted saturated, unsaturated or aromatic heterocyclic radical having from 5 to 10 atoms that can contain 1, 2 or 3 heteroatoms selected from O, N and S; a —COOR₁₁ radical; a —CONR₁₂R₁₃ radical; an alkoxy radical optionally substituted having from 1 to 18 carbon atoms; an optionally substituted aryloxy radical; an optionally substituted —COR₁₄ radical; a C₃-C₆ cycloalkyl radical; a linear or branched chain, saturated or unsaturated alkyl radical that contains from 1 to 18 carbon atoms, optionally substituted with at least one hydroxyl radical, an SO₃M or —N(R₁₅)₃⁺ group or else a group of general formula (VI)

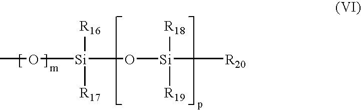

in which
m=0 or 1;
p=0, 1, 2, 3, or 4;
R₁₆, R₁₇, R₁₈, R₁₉ and R₂₀ are same as or different from each other and represent an optionally substituted alkyl radical having from 1 to 6 carbon atoms; an alkoxy radical of 1 to 6 atoms of carbon; an optionally substituted aryl radical or an —OSi(R₂₁)₃ radical;
R₂₁ represents an alkyl radical having from 1 to 6 carbon atoms, an alkoxy radical having from 1 to 6 carbon atoms or an optionally substituted aryl radical;

R₁₁, R₁₂ and R₁₃ are same as or different from each other and represent an atom of hydrogen, or an optionally substituted linear or branched chain alkyl radical having from 1 to 18 carbon atoms; a C₃-C₆ cycloalkyl radical; or else

R₁₂ and R₁₃ form together with the nitrogen atom a saturated heterocylic compound having from 5 to 7 carbon atoms that can contain 1, 2 or 3 heteroatoms selected from O, N and S, optionally substituted;

R₁₄ is an optionally substituted alkyl radical or an optionally substituted aryl radical;

R₁₅ is an optionally substituted alkyl radical;

M is H, Na or K;

R₆ and R₇ or else, R₆ and R₁₄ are condensates forming with the phenyl a polycyclic system having from 9 to 15 atoms, optionally substituted;

R₈ and R₉ can be same as or different from each other and represent an atom of hydrogen; an optionally substituted acyl radical having from 1 to 18 carbon atoms; a linear or branched chain, saturated or unsaturated alkyl radical that contains from 1 to 18 carbon atoms, optionally substituted with at least one hydroxyl radical, an SO₃M or —N(R₁₅)₃⁺ group or else a group of general formula (VI), as defined above;

R₁₀ represents an atom of hydrogen or an SO₃M radical, with M being as defined above.

In another aspect this invention relates to compounds of formula (I) wherein

n=1, 2, 3 or 4, specially n=1;

R₁ represents an atom of hydrogen; a linear or branched alkyl radical having from 1 to 3 atoms, optionally substituted; or a substituted R₂′, R₃′ phenyl radical;

R₂, R₂′, R₃ and R₃′ are the same as or different from each other and represent an atom of hydrogen; an optionally substituted linear or branched alkyl radical having from 1 to 3 carbon atoms; an alkoxy radical having from 1 to 3 carbon atoms; an aryl radical; halogen or hydroxyl radical or else

R₂ and R₃ are condensates that form with the phenyl a naphthalene ring, optionally substituted;

R₄ and R₅ are the same as or different from each other and represent an atom of hydrogen; an optionally substituted linear or branched alkyl radical having from 1 to 4 carbon atoms; or an optionally substituted aryl radical;

A₁ is a radical of general formula (II), (III) or (IV)

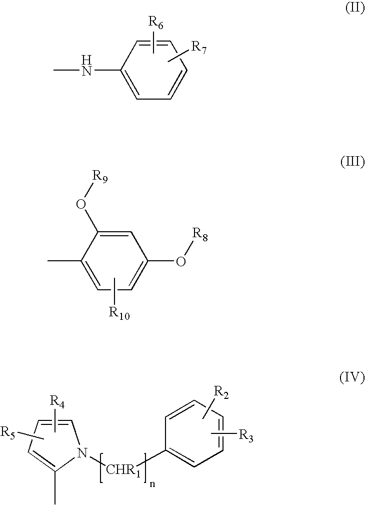

A₂ is a radical of general formula (II) or (V)

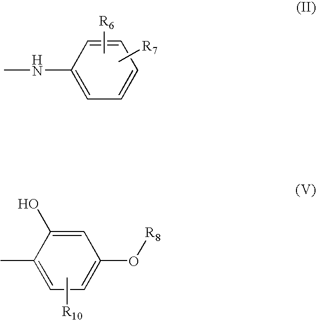

R₆ represents an atom of hydrogen; an optionally substituted, linear or branched chain, saturated or unsaturated alkyl radical that contains from 1 to 6 carbon atoms; or a hydroxyl radical;

R₇ represents an atom of hydrogen; an optionally substituted aryl radical; an optionally substituted saturated, unsaturated or aromatic heterocyclic radical having from 5 to 10 atoms that can contain 1, 2 or 3 heteroatoms selected from O, N and S; a —COOR₁₁ radical; a —CONR₁₂R₁₃ radical; an alkoxy radical having from 1 to 18 carbon atoms optionally substituted; an optionally substituted aryloxy radical; an optionally substituted —COR₁₄ radical; a C₃-C₆ cycloalkyl radical; a linear or branched chain, saturated or unsaturated alkyl radical that contains from 1 to 18 carbon atoms, optionally substituted with at least one hydroxyl radical, an SO₃M or —N(R₁₅)₃⁺ group or else a group of general formula (VI)

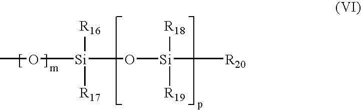

in which
m=0 or 1;
p=0, 1, 2, 3 or 4;

R₁₆, R₁₇, R₁₈, R₁₉ and R₂₀ are the same as or different from each other and represent an optionally substituted alkyl radical having from 1 to 6 carbon atoms; an alkoxy radical having from 1 to 6 carbon atoms, an optionally substituted aryl radical or an —OSi(R₂₁)₃ radical;

R₂₁ represents an alkyl radical of 1 to 6 carbon atoms, un alkoxy radical of 1 to 6 atoms of carbon or an optionally substituted aryl radical;

R₁₁, R₁₂ and R₁₃ are the same as or different from each other and represent an atom of hydrogen; an alkyl radical, linear or branched chain having from 1 to 18 carbon atoms optionally substituted; a C₃-C₆ radical cycloalkyl, or else

R₁₂ and R₁₃ form together with the nitrogen atom a saturated heterocylic compound having from 5 to 7 carbon atoms that can contain 1, 2 or 3 heteroatoms selected from O, N and S;

R₁₄ is an optionally substituted alkyl radical or an optionally substituted aryl radical;

R₁₅ is an optionally substituted alkyl radical;

M is H, Na or K;

R₆ and R₇ or else, R₆ and R₁₄ are condensates forming with the phenyl a polycyclic system having from 9 to 15 atoms, optionally substituted;

R₈ and R₉ can be the same as or different from each other and represent an atom of hydrogen; an optionally substituted acyl radical having from 1 to 18 carbon atoms; a linear or branched, saturated or unsaturated alkyl radical that contains from 1 to 18 carbon atoms, optionally substituted with at least one hydroxyl radical, an SO₃M or —N(R₁₅)₃⁺ group or else a group of general formula (VI), as defined above;

R₁₀ represents an atom of hydrogen or a SO₃M radical, with M being as defined above.

In an other aspect this invention relates to compounds of general formula (I) wherein n=1 and the other radicals are as defined above.

In the present invention, the term “optionally substituted”—if not defined otherwise—means a radical that can be substituted in at least one position, the substituent being a C₁-C₆ alkyl radical; C₂-C₆ alkenyl; aryl; saturated, unsaturated or aromatic heterocycle having from 5 to 10 atoms that can contain 1, 2 or 3 heteroatoms selected from O, N and S; a hydroxyl radical; C₁-C₈ alkoxide; or a halogen such as chlorine or fluorine.

In a preferred embodiment of the first aspect of the invention, A₁ and A₂ are the same and represent a radical of general formula (II) or one of general formula (V)

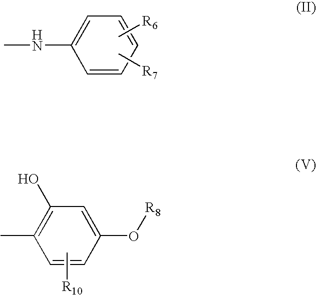

where R₆, R₇, R₈ and R₁₀ are as defined above.

In another preferred embodiment of the first aspect of the invention, the pyrrolyltriazine derivative corresponds to general formula (IA):

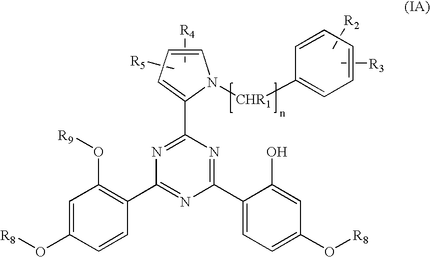

where R₁-R₅, R₈-R₉ and n are as defined above.

In another preferred embodiment of the first aspect of the invention, the pyrrolyltriazine derivative corresponds to general formula (IB):

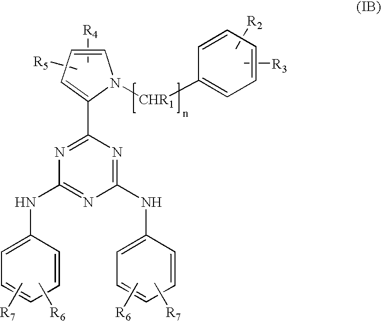

where R₁-R₇ and n are as defined above.

In another preferred embodiment of the first aspect of the invention, R₁ represents hydrogen, alkyl, phenyl and phenylalkyl, optionally substituted in at least one position by a phenyl, chloro, bromo, fluoro, alkoxy or alkyl group.

In another preferred embodiment, R₂ and R₂′ represent hydrogen, phenyl, methyl or ethyl.

In yet another embodiment R₃ and R₃′ represent hydrogen, phenyl, methyl or ethyl.

In another preferred embodiment, R₂ and R₃ form a naphthalene group.

In another preferred embodiment, R₄ and R₅ are same as or different from each other and represent hydrogen, methyl or phenyl.

In another preferred embodiment R₆ represents hydrogen, hydroxyl, methyl or ethyl.

In another preferred embodiment R₇ represents hydrogen, hydroxyl, methyl, ethyl, tert-butyl, benzyl, cyclohexyl, ethoxyphenyl, biphenyl, —COOR₁₁, —CONR₁₂R₁₃, —COR₁₄ or the group of general formula (VI):

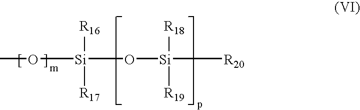

In another preferred embodiment, R₈ and R₉ are same as or different from each other and represent ethylhexyl or a linear or branched, saturated or unsaturated alkyl radical that contains from 1 to 18 carbon atoms, optionally substituted with at least one —SO₃M or —N(R₁₅)₃⁺ group.

In another preferred embodiment, R₁₁ represents hydrogen, methyl, ethyl, propyl, butyl, tert-butyl, pentyl, hexyl or 2-ethylhexyl.

In another preferred embodiment, R₁₂ represents hydrogen, methyl, ethyl, propyl, butyl, tert-butyl, pentyl, hexyl or 2-ethylhexyl.

In another preferred embodiment, R₁₃ represents hydrogen, methyl, ethyl, propyl, butyl, tert-butyl, pentyl, hexyl or 2-ethylhexyl.

In another preferred embodiment, R₁₄ represents methyl, ethyl, propyl, butyl, ter-butyl or phenyl.

In yet another preferred embodiment R₁₆ to R₂₀ represent methyl, ethyl, methoxy, ethoxy or phenyl.

In another embodiment still, R₂₁ represents methyl, ethyl, methoxy, ethoxy or phenyl.

A₁ and A₂ are selected according to any of the definitions mentioned above.

Advantageously, said derivative of general formula (I) according to the first aspect of the invention is selected from the group that consists in:
2-(1-benzyl-1H-pyrrol-2-yl)-4,6-bis(2,4-dihydroxyphenyl)-1,3,5-triazine;
2-(1-benzyl-1H-pyrrol-2-yl)-4,6-bis[4-(2-ethylhexyloxy)-2-hydroxyphenyl]-1,3,5-triazine;
2-(1-benzyl-1H-pyrrol-2-yl)-4,6-bis[4-(butoxycarbonyl)phenylamino]-1,3,5-triazine;
2-(1-benzyl-1H-pyrrol-2-yl)-4,6-bis(biphenyl-4-ylamino)-1,3,5-triazine;
2-(1-benzyl-1H-pyrrol-2-yl)-4,6-bis(4-benzoylphenylamino)-1,3,5-triazine;
2-(1-benzyl-1H-pyrrol-2-yl)-4,6-bis(9-oxo-9H-fluoren-3-ylamino)-1,3,5-triazine;
2-(1-benzyl-1H-pyrrol-2-yl)-4,6-bis[4-(imidazo[1,2-a]pyridin-2-yl)phenylamino]-1,3,5-triazine;
2-(1-benzydryl-1H-pyrrol-2-yl)-4,6-bis[4-(butoxycarbonyl)phenylamino]-1,3,5-triazine.

Surprisingly, the inventors of the present invention have found that the pyrrolyltriazine derivatives of general formula (I) absorb in the ultraviolet radiation range of both types A and B, with said derivatives therefore being useful as UV radiation absorbing agents and effective simultaneously in protecting against UV-A and UV-B radiation.

Another aspect of the present invention is the methods to prepare a pyrrolyltriazine derivatives according to the first aspect of the invention.

In particular, the pyrrolyltriazine derivatives of general formula I, in which A₁ is a radical of general formula (III) and A₂ is a radical of general formula (V) are as defined above, with R₁₀ being hydrogen:

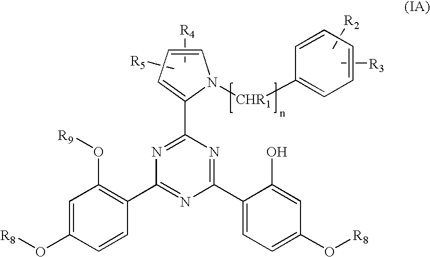

in which R₁, R₂, R₃, R₄, R₅, R₈, R₉ and n have the meaning indicated above, and can be prepared as indicated in Reaction Scheme 1.

The process described in this Reaction Scheme 1 has shown very good possibilities in order to obtain industrial quantities of compounds and leading also to compounds of general formula (IA) that show a very good stability and will also give a very good protection against UV-A and UV-B radiation, especially UV-A radiation.

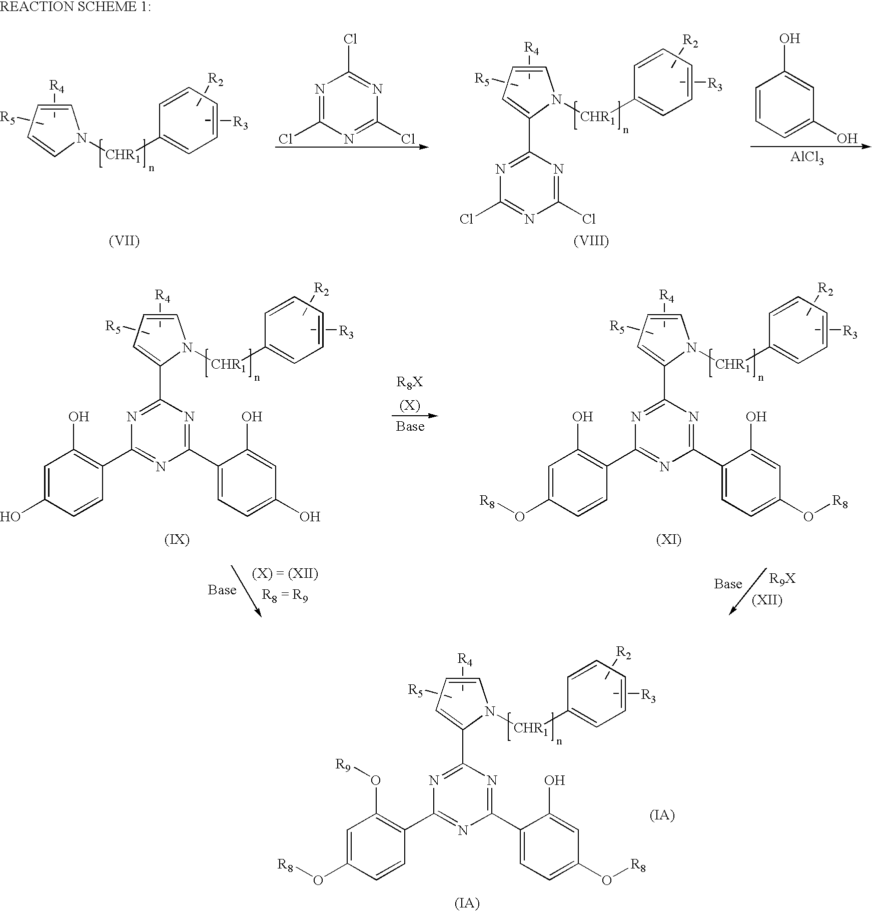

Briefly, the compounds of general formula (VIII) are prepared as described in U.S. Pat. No. 5,955,060 and Chakrabarti, J. K. and Tupper, D. E., J. Het. Chem. 1974, 11 (3), 417-421.

The compound of general formula (IX) is obtained by Friedel-Crafts acylation of resorcinol with the compound of general formula (VIII) in the presence of a Lewis acid, in particular aluminium chloride, in an inert solvent such as xylene (mixture of isomers) and at a temperature between 60° C. and 100° C., in accordance with the process described in U.S. Pat. No. 5,955,060.

The esterification of the p-hydroxyl groups that leads the compounds of general formula (XI) is carried out by alkylation of the compounds of general formula (IX) with a compound of general formula (X), where R₈ is as defined above, and X is a leaving group such as chloro, bromo, tosyl or mesyl, in the presence of a base, such as sodium hydroxide, cesium carbonate, potassium carbonate, sodium tert-butoxide and potassium tert-butoxide, in an appropriate polar solvent such as 2-methoxyethanol, 2-ethoxyethanol, N-methylpyrrolidone, N,N-dimethylformamide and ethanol, at a temperature that ranges between 80° C. and 120° C.

The trialkylated compounds of general formula (IA) are obtained by alkylation of the compound of general formula (XI) with a compound of general formula (XII), in which R₉ and X are as defined above, in the presence of a base, such as potassium carbonate, cesium carbonate, sodium hydroxide, potassium hydroxide, sodium tert-butoxide and potassium tert-butoxide, in a polar solvent such as 2-methoxyethanol, 2-ethoxyethanol, N-methylpyrrolidone, N,N-dimethylformamide and ethanol, and at a temperature that ranges between 120° C. and the boiling temperature of the solvent.

When the R₈ and R₉ radicals are the same, the corresponding compound of general formula (IA) is obtained directly from the compound of general formula (IX).

The compounds of general formula (I) in which R₁₀ is —SO₃M, with M as defined above, can be obtained, for example, following the procedures described in U.S. Pat. No. 6,090,370, in particular column 5, line 59-column 6, line 8.

The compounds of general formula (I), having an —SO₃M group in an alkyl chain, and with M as defined above, can be obtained, for example, following the procedures described in Lewin, G. et al., J. Nat. Prod., 58 (1995) 12, 1840-1847.

The compounds of general formula (I), having a —N(R₁₅)₃⁺ group in an alkyl chain, with R₁₅ as defined above, can be obtained, for example, following the procedures described in Sharma, M. L. et al., J. Indian Chem. Soc., 74 (1997)4, 343-344.

In another embodiment of the process, the triazine derivatives of general formula (I) in which A₁ and A₂ are the same and correspond to a radical of general formula (II):

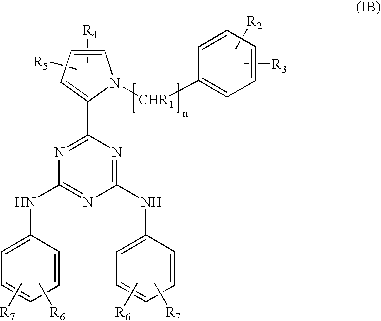

and where R₁, R₂, R₃, R₄, R₅, R₆, R₇ and n have the meaning stated above, can be prepared as shown in Reaction Scheme 2.

The process described in this Reaction Scheme 2 has also shown very good possibilities in order to obtain industrial quantities of compounds of general formula (IB).

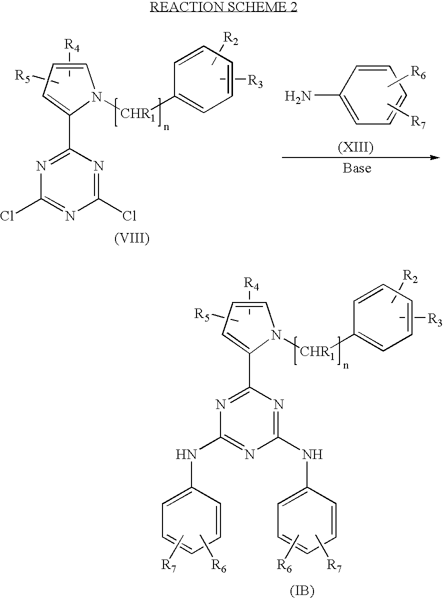

Briefly, the compound of general formula (VIII), prepared as defined above, reacts with at least 2 equivalents of aniline of general formula (XIII), with R₆ and R₇ as defined above, in the presence of a base such as N,N-diisopropylethylamine, potassium carbonate, sodium carbonate, cesium carbonate, sodium hydroxide or potassium hydroxide in a solvent such as dioxane, toluene, xylene (mixture of isomers), N,N-dimethylformamide, N-ethylpyrrolidone or acetone, at a temperature that ranges between 0° C. and the boiling temperature of the solvent, preferably between ambient temperature and the boiling temperature of the solvent, and more preferably between 50° C. and the boiling temperature of the solvent.

As indicated above, the pyrrolyltriazine derivatives of general formula (I) according to the first aspect of the present invention have physico-chemical properties such as absorption of ultraviolet light, which permits their use as protecting agents against UV radiation.

Also object of the present invention, therefore, are cosmetic, dermatological or pharmaceutical formulations that include one or more derivatives of general formula (I), according to the first aspect of the invention, and at least one cosmetically, dermatologically or pharmaceutically acceptable carrier or excipient.

In a preferred embodiment, said cosmetic, dermatological or pharmaceutical formulation also includes at least one organic, micronised organic or inorganic filter against solar radiation.

In another preferred embodiment, said formulation also includes at least one active substance.

Said cosmetic, dermatological or pharmaceutical formulation can be adapted to apply to the skin and lips in the form of: a non-ionic vesicular dispersion, emulsion, cream, lotion, gel, aerosol, cream-gel, gel-cream, suspension, dispersion, powder, solid stick, foam, spray, oil, ointment or fluid, among others.

Similarly, said formulation can be adapted to apply to the hair in the form of a shampoo, lotion, gel, fluid, lacquer, foam, dye, emulsion, cream or spray, among others, and on the nails in the form of a nail varnish, oil or gel, among others.

Moreover, the organic, micronised organic and inorganic filters are selected from those acceptable under the country's legislation.

For example, the organic filters can be selected from those approved by the Council of the European Community (revised text of European Directive 76/768/EEC Annex-7. pages 76-81, published on Oct. 15, 2003) and by the U.S. Food and Drug Administration (see, for example, “Food and Drugs, Sunscreen drug products for over-the-counter human use”, title 21, volume 5 of the Code of Federal Regulations, revised on 1 Apr. 2004), such as antranilates; camphor derivatives; dibenzoylmethane derivatives; benzotriazole derivatives; diphenylacrylate derivatives; cinnamic derivatives; salicylic derivatives; triazine derivatives such as those described in patents EP-863145, EP-517104, EP-570838, EP-796851, EP-775698 and EP-878469. benzophenone derivatives; benzalmalonate derivatives; benzimidazol, imidizoline derivatives; p-aminobenzoic acid derivatives; polymeric and silicone filters.

The inorganic filters can be selected from a group that comprises: metallic oxides and treated and untreated pigments, nanopigments, such as titanium dioxide (amorphous or crystalline), iron oxides, zinc, zirconium or cerium. In addition, alumina and/or aluminium stearate are conventional coating agents. Examples of untreated metallic oxides as (non-coated) inorganic filters are those described in patent applications EP518772 and EP518773.

The cosmetic, dermatological and pharmaceutical formulations of the present invention can additionally contain additives and adjuvants that can be selected from fatty acids, organic solvents, thickening agents, softening agents, antioxidants, opacifiers, stabilisers, emollients, hydroxy-acids, antifoaming agents, moisturizing agents, vitamins, fragrances, preservatives, surfactants, sequestering agents, polymers, propellants, acidifying or basifying agents, colorants, dyes, dihydroxyacetone, insect repellent or any other ingredient commonly used in cosmetic formulations, and in particular in the production of photoprotective compositions.

Examples of substances/fatty acids include, among others, oils or waxes or mixtures thereof and they can include fatty acids, fatty alcohols and fatty acid esters. The oils are selected, advantageously, from animal, vegetable or synthetic oils, and in particular from liquid petrolatum, liquid paraffin, volatile and non-volatile silicone oils, isoparaffins, polyalphaolefins, or fluorinated or perfluorinated oils. Similarly, the waxes are selected, advantageously, from animal, vegetable, mineral or synthetic waxes well known to the skilled in the art.

Examples of organic solvents include short alcohols and polyols.

The thickeners are selected, advantageously, from acrylic-acid cross-linked polymers, modified and unmodified locust bean gums, celluloses and xanthan gums, such as hydroxypropylated locust bean gums, methylhydroxyethylcellulose, hydroxypropylmethylcellulose or hydroxyethylcellulose.

When selecting the excipients, adjuvants, etc., the skilled in the art will ensure that they do not affect the activity of the pyrrolyltriazine derivatives of general formula (I) in accordance with the invention.

A fourth aspect, the present invention relates to the use of a pyrrolyltriazine derivatives according to the first aspect of the invention in a cosmetic, dermatological or pharmaceutical formulation as a UV radiation filtering agent.

A fifth aspect, the present invention relates to the use of a pyrrolyltriazine derivatives according to the first aspect of the invention for manufacturing a formulation for protection of the skin, lips and/or related tissues of a mammal against solar radiation.

A sixth aspect, the present invention relates to the use of at least one derivative of pyrrolyltriazine according to the first aspect of the invention for manufacturing a formulation for preventive use, as a coadjuvant in the treatment of pathologies caused by ultraviolet radiation on the skin, lips and/or related tissues of a mammal, such as polymorphous light eruptions, photoageing, actinic keratasis, vitiligo, solar urticaria, chronic actinic dermatitis and xeroderma pigmentosum. Preferably, said formulation is applied topically.

In a preferred embodiment said mammal is a human being.

The properties of the pyrrolyltriazine derivatives of general formula (I) make these compounds also useful as photostabilisers of synthetic polymers and as solar filters for textile fibres.

There follow some examples by way of non-restrictive illustration of the present invention.

### EXAMPLES

### Example 1

### Synthesis of 2-(1-benzyl-1H-pyrrol-2-yl)-4,6-bis[4-(2-ethylhexyloxy)-2-hydroxyphenyl]-1,3,5-triazine

### a) Synthesis of 2-(1-benzyl-1H-pyrrol-2-yl)-4,6-dichloro-1,3,5-triazine

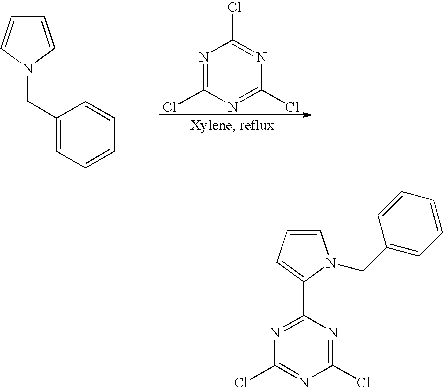

A mixture of 1-benzylpyrrol (5.0 g, 31.8 mmol) and 2,4,6-trichloro-1,3,5-triazine (6.8 g, 36.9 mmol) is refluxed in xylene (35 mL) for 26 hours. The solvent is evaporated to dryness, the crude product is cooled and methanol (35 mL) is added at room temperature and the mixture left under stirring for 25 min. The solid obtained is filtered, washed with methanol and dried to obtain 2-(1-benzyl-1H-pyrrol-2-yl)-4,6-dichloro-1,3,5-triazine (8.35 g, 27.4 mmol, 86%, m.p.=150-151° C.).

¹H NMR (300 MHz, CDCl₃): δ 5.72 (s, 2H), 6.36 (dd, J=4.1 Hz, J′=2.5 Hz, 1H), 7.09 (d, J=6.7 Hz, 2H), 7.15 (m, 1H), 7.23-7.33 (m, 3H), 7.59 (dd, J=4.1 Hz, J′=1.8 Hz, 1H).

### b) Synthesis of 2-(1-benzyl-1H-pyrrol-2-yl)-4,6-bis(2,4-dihydroxyphenyl)-1,3,5-triazine

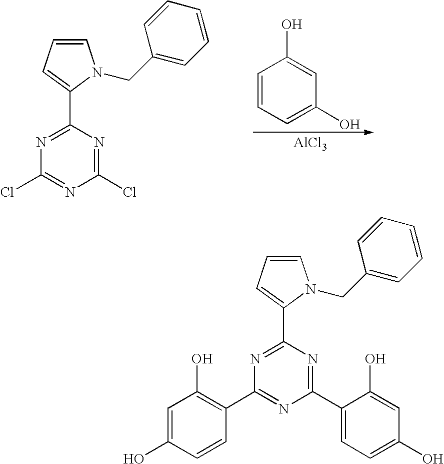

To a mixture of resorcinol (2.2 g, 20 mmol) in xylene (50 mL) heated to 50° C. is added 2-(1-benzyl-1H-pyrrol-2-yl)-4,6-dichloro-1,3,5-triazine (3 g, 9.8 mmol) and aluminium trichloride (2.6 g, 19.5 mmol) and kept at 80-85° C. for 3 hours. The mixture is cooled, the xylene is decanted, HCl 2N (50 mL) is added and left under stirring. The solid obtained is filtered, washed with HCl 2N and water and dried. The resulting crude product is treated with acetone and the solid is filtered and dried to obtain 2-(1-benzyl-1H-pyrrol-2-yl)-4,6-bis(2,4-dihydroxyphenyl)-1,3,5-triazine (4.6 g, quantitative yield, m.p.>275° C.).

¹H NMR (300 MHz, DMSO-d₆): δ 5.95 (s, 2H), 6.30-6-46 (m, 5H), 7.03 (d, J=6.5 Hz, 2H), 7.18 (m, 1H), 7.23 (m, 3H), 7.42 (s, 1H), 7.83 (d, J=8.7 Hz, 2H).

### c) Synthesis of 2-(1-benzyl-1H-pyrrol-2-yl)-4,6-bis[4-(2-ethylhexyloxy)-2-hydroxyphenyl]-1,3,5-triazine

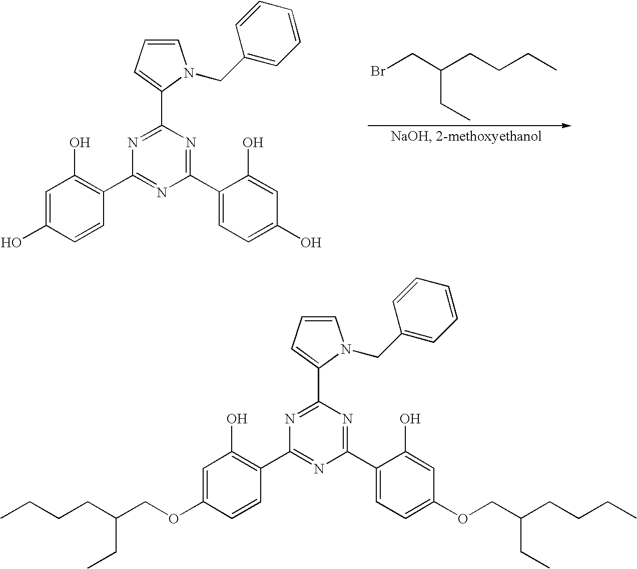

To a mixture of 2-(1-benzyl-1H-pyrrol-2-yl)-4,6-bis(2,4-dihydroxyphenyl)-1,3,5-triazine (2.3 g, 5.1 mmol) and a 30% solution of NaOH (1.5 g, 11.2 mmol) in 2-ethoxyethanol (40 mL) heated to 80° C., a solution of 3-(bromomethyl)heptane (2.1 g, 10.8 mmol) in 2-methoxyethanol (8 mL) is added slowly. Once the addition is finished, it is heated to 110-115° C. for 16 hours following the reaction by TLC. The mixture is cooled to 70-80° C. and a 30% solution of NaOH (1.5 g, 11.2 mmol) and 3-(bromomethyl)heptane (2.1 g, 10.8 mmol) in 2-methoxyethanol (8 mL) are added again and heated to 110-115° C. for 8 hours. The solvent is evaporated at reduced pressure and the residue is diluted in ethylic ether. The organic phase is washed with a dilute solution of acetic acid and a dilute solution of NaHCO₃ and evaporated at reduced pressure. The resulting crude product is purified by silica gel column chromatography to obtain 2-(1-benzyl-1H-pyrrol-2-yl)-4,6-bis[4-(2-ethylhexyloxy)-2-hydroxyphenyl]-1,3,5-triazine (1.2 g, 1.77 mmol, 35%, m.p.=66-68° C., as a white solid).

¹H NMR (300 MHz, DMSO-d₆): δ 0.93 (m, 12H), 1.28-1.66 (m, 16H), 1.75 (m, 2H), 3.90 (d, J=5.7 Hz, 4H), 5.94 (s, 2H), 6.38 (dd, J=4.0 Hz, J′=2.6 Hz, 1H), 6.42-6.54 (m, 4H), 7.03 (m, 1H), 7.09 (d, J=6.9 Hz, 2H), 7.20-7.34 (m, 3H), 7.40 (m, 1H), 7.93 (d, J=7.6 Hz, 2H), 13.56 (m, 2H).

λₘₐₓ=350-352 nm εₘₐₓ=66000 M⁻¹ cm⁻¹ (chloroform)

### Example 2

### Synthesis of 2-(1-benzyl-1H-pyrrol-2-yl)-4,6-bis[4-(butoxycarbonyl)phenylamino]-1,3,5-triazine

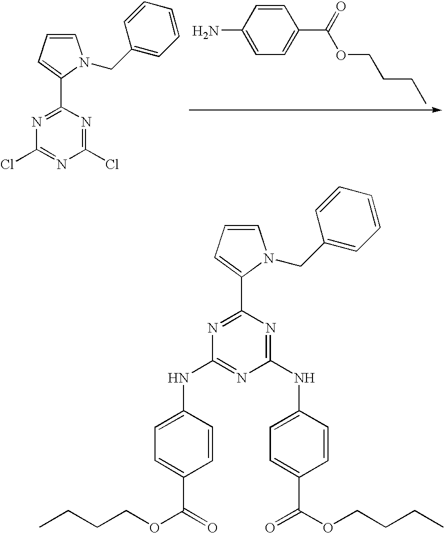

A mixture of 2-(1-benzyl-1H-pyrrol-2-yl)-4,6-dichloro-1,3,5-triazine (150 mg, 0.49 mmol), butyl 4-aminobenzoate (190 mg, 0.98 mmol) and potassium carbonate (136 mg, 0.98 mmol) in dioxane (10 mL) is refluxed for 5 hours, following the reaction by TLC. The solvent is evaporated at reduced pressure, the residue is diluted in a mixture of ethyl acetate and ethyl ether and washed with water. The organic phase is separated, dried and evaporated. The resulting crude product is purified by silica gel column chromatography to obtain 2-(1-benzyl-1H-pyrrol-2-yl)-4,6-bis[4-(butoxycarbonyl)phenylamino]-1,3,5-triazine (98 mg, 0.16 mmol, 32%, m.p.=173-174° C.).

¹H NMR (300 MHz, CDCl₃): δ 0.99 (t, J=7.3 Hz, 3H), 1.49 (m, 2H), 1.78 (m, 2H), 4.32 (t, J=6.6 Hz, 2H), 5.82 (s, 2H), 6.32 (m, 1H), 6.93 (s, 1H), 6.95 (m, 2H), 7.29 (m, 3H), 7.49 (m, 1H), 7.63 (d, J=8.5 Hz, 4H), 7.99 (d, J=8.5 Hz, 4H).

UV λₘₐₓ=312 nm; εₘₐₓ=64000 M⁻¹ cm⁻¹ (chloroform)

### Example 3

### Synthesis of 2-(1-benzyl-1H-pyrrol-2-yl)-4,6-bis(biphenyl-4-ylamino)-1,3,5-triazine

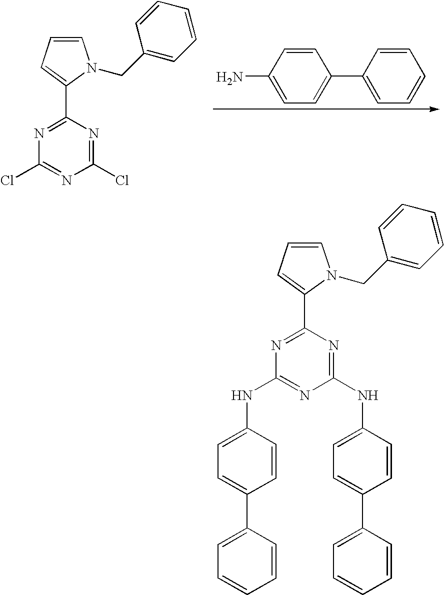

A mixture of 2-(1-benzyl-1H-pyrrol-2-yl)-4,6-dichloro-1,3,5-triazine (92 mg, 0.30 mmol), 4-aminobiphenyl (110 mg, 0.65 mmol) and potassium carbonate (100 mg, 0.72 mmol) in dioxane (4 mL) is refluxed for 4 hours, following the reaction by TLC. The solvent is evaporated at reduced pressure, water is added to the residue and the precipitate is filtered. The solid obtained is digested in hot MeOH, and is cooled and filtered to obtain 2-(1-benzyl-1H-pyrrol-2-yl)-4,6-bis(biphenyl-4-ylamino)-1,3,5-triazine (97 mg, 0.17 mmol, 57%, m.p.=185-186° C.).

¹H NMR (300 MHz, DMSO-d₆): δ 5.97 (br s, 2H), 6.22 (m, 1H), 7.02 (m, 2H), 7.14-7.34 (m, 7H), 7.44 (m, 4H), 7.63 (m, 8H), 7.82 (m, 4H), 9.69 (br s, 2H).

UV λₘₐₓ=309 nm; εₘₐₓ=73000 M⁻¹ cm⁻¹ (ethanol)

### Example 4

### Synthesis of 2-(1-benzyl-1H-pyrrol-2-yl)-4,6-bis(4-benzoylphenylamino)-1,3,5-triazine

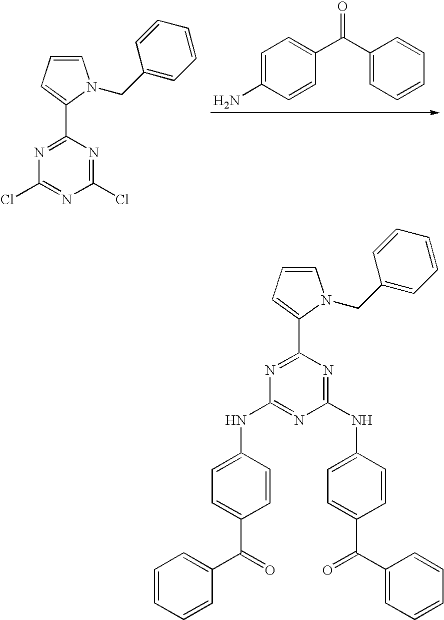

A mixture of 2-(1-benzyl-1H-pyrrol-2-yl)-4,6-dichloro-1,3,5-triazine (153 mg, 0.50 mmol), 4-aminobenzophenone (200 mg, 1.01 mmol) and potassium carbonate (200 mg, 1.45 mmol) in dioxane (8 mL) is refluxed for 6 hours, following the reaction by TLC. The solvent is evaporated at reduced pressure and the residue is diluted in ethyl ether and washed with dilute HCl. The organic phase is evaporated to dryness and the residue crystallised with MeOH to obtain 2-(1-benzyl-1H-pyrrol-2-yl)-4,6-bis(4-benzoylphenylamino)-1,3,5-triazine (40 mg, 0.06 mmol, 12%, m.p.=115-118° C.)

¹H NMR (300 MHz, CDCl₃): δ 5.86 (s, 2H), 6.32 (m, 1H), 6.93 (s, 1H), 7.02 (m, 2H), 7.18-7.30 (m, 3H), 7.40-7.53 (m, 5H), 7.59 (m, 2H), 7.66-7.82 (m, 12H).

UV λₘₐₓ=330 nm; εₘₐₓ=80000 M⁻¹ cm⁻¹ (ethanol)

### Example 5

### Synthesis of 2-(1-benzyl-1H-pyrrol-2-yl)-4,6-bis(9-oxo-9H-fluoren-3-ylamino)-1,3,5-triazine

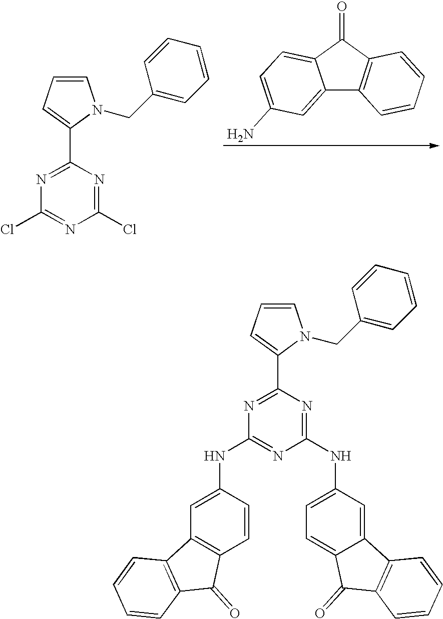

A mixture of 2-(1-benzyl-1H-pyrrol-2-yl)-4,6-dichloro-1,3,5-triazine (153 mg, 0.50 mmol), 3-amino-9H-fluoren-9-one (200 mg, 1.02 mmol) and potassium carbonate (200 mg, 1.45 mmol) in dioxane (8 mL) is refluxed for 6 hours, following the reaction by TLC. The reaction is cooled and the precipitate is filtered and washed with AcOEt. The solid obtained is recrystallised from EtOH to obtain 2-(1-benzyl-1H-pyrrol-2-yl)-4,6-bis(9-oxo-9H-fluoren-3-ylamino)-1,3,5-triazine (160 mg, 0.26 mmol, 52%, m.p.=140-142° C.)

¹H NMR (300 MHz, CDCl₃): δ 5.80 (s, 2H), 6.30 (m, 1H), 6.89 (m, 1H), 6.96 (m, 2H), 7.10-7.70 (m, 16H), 7.95 (m, 2H).

UV λₘₐₓ=292 nm; εₘₐₓ=77000 M⁻¹ cm⁻¹ (ethanol).

### Example 6

### Synthesis of 2-(1-benzyl-1H-pyrrol-2-yl)-4,6-bis[4-(imidazo[1,2-a]pyridin-2-yl)phenylamino]-1,3,5-triazine

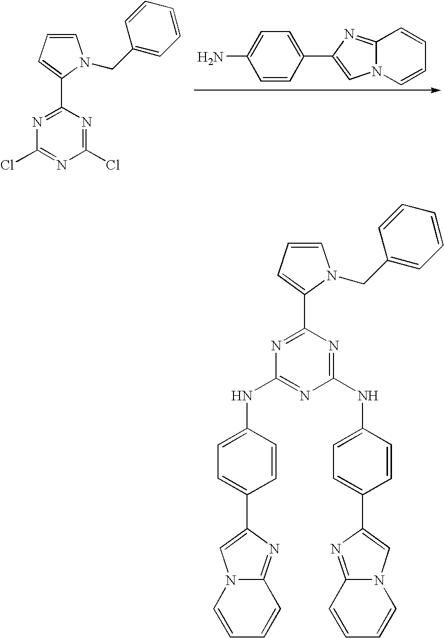

A mixture of 2-(1-benzyl-1H-pyrrol-2-yl)-4,6-dichloro-1,3,5-triazine (150 mg, 0.49 mmol), 4-(imidazo[1,2-a]pyridin-2-yl)phenylamine (210 mg, 1.0 mmol) and N,N-diisopropylethylamine (130 mg, 1.0 mmol) in N-methylpyrrolidone (3 mL) is heated at 115-120° C. for 5 hours, following the reaction by TLC. The mixture is cooled and then poured dropwise onto water. The precipitate is filtered, washed with water and purified by silica gel chromatography. The residue obtained from evaporation of the fractions is treated with ethanol (4 mL) and acetone (3 mL), to crystallise 2-(1-benzyl-1H-pyrrol-2-yl)-4,6-bis[4-(imidazo[1,2-a]pyridin-2-yl)phenylamino]-1,3,5-triazine (115 mg, 0.18 mmol, 37%, m.p.=147-148° C.).

¹H NMR (300 MHz, DMSO-d₆): δ 5.98 (br s, 2H), 6.23 (m, 1H), 6.87 (m, 2H), 7.03 (m, 2H), 7.12-7.29 (m, 7H), 7.56 (d, J=9.1 Hz, 2H), 7.72-7.94 (m, 8H), 8.32 (s, 2H), 8.50 (d, J=6.7 Hz, 2H), 9.67 (br s, 2H).

UV λₘₐₓ=332 nm εₘₐₓ=71000 M⁻¹ cm⁻¹ (ethanol)

### Example 7

### Synthesis of 2-(1-benzydryl-1H-pyrrol-2-yl)-4,6-bis[4-(butoxycarbonyl)phenylamino]-1,3,5-triazine

### a) Synthesis of 2-(1-benzydryl-1H-pyrrol-2-yl)-4,6-dichloro-1,3,5-triazine

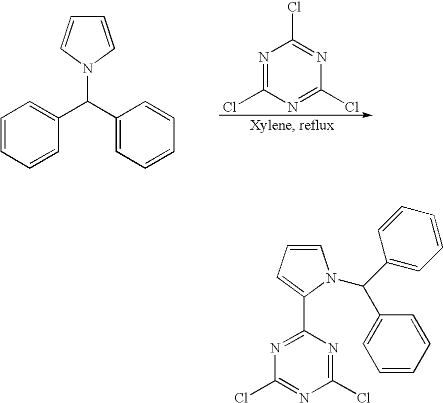

A mixture of de 1-benzydryl-1H-pyrrol (1.8 g, 7.7 mmol) and 2,4,6-trichloro-1,3,5-triazine (1.7 g, 9.2 mmol) in xylene (50 mL) is refluxed for 14 hours, following the reaction by TLC. The solvent is evaporated to dryness, petroleum ether is added and the solid obtained is filtered, washed with petroleum ether and methanol to obtain 2-(1-benzydryl-1H-pyrrol-2-yl)-4,6-dichloro-1,3,5-triazine (1.5 g, 3.9 mmol, 51%, m.p.=127-129° C., yellowish solid).

¹H NMR (300 MHz, CDCl₃): δ 6.29 (dd, J=4.1 Hz, J′=2.6 Hz, 1H), 6.82 (m, 1H), 7.07 (m, 4H), 7.29-7.37 (m, 6H), 7.65 (dd, J=4.1 Hz, J′=1.8 Hz, 1H), 8.01 (s, 1H).

### b) Synthesis of 2-(1-benzydryl-1H-pyrrol-2-yl)-4,6-bis[4-(butoxycarbonyl)phenylamino]-1,3,5-triazine

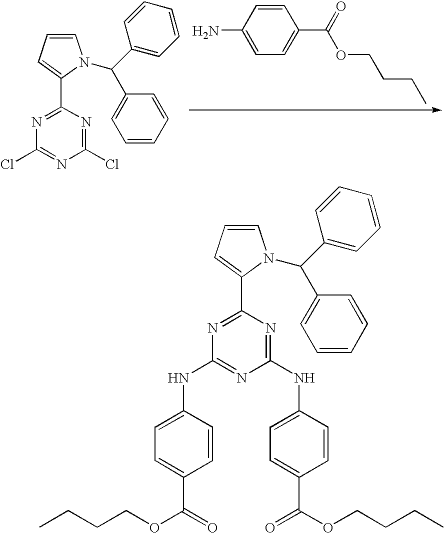

A mixture of 2-(1-benzydryl-1H-pyrrol-2-yl)-4,6-dichloro-1,3,5-triazine (100 mg, 0.26 mmol), butyl 4-aminobenzoate (101 mg, 0.52 mmol) and N,N-diisopropylethylamine (135 μL, 0.78 mmol) in dioxane (5 mL) is refluxed for 16 hours, following the reaction by TLC. The solvent is evaporated at reduced pressure, the residue is dissolved in ethyl acetate and washed with saturated solution of NaCl. The organic phase is separated, dried and evaporated. The resulting crude product is purified by silica gel column chromatography to obtain 2-(1-benzydryl-1H-pyrrol-2-yl)-4,6-bis[4-(butoxycarbonyl)phenylamino]-1,3,5-triazine (150 mg, 0.22 mmol, 83%, m.p.=80-82° C., white solid).

¹H NMR (300 MHz, CDCl₃): δ 0.98 (t, J=7.4 Hz, 6H), 1.48 (m, 4H), 1.72 (m, 4H), 4.30 (t, J=6.6 Hz, 4H), 6.25 (m, 1H), 6.71 (m, 1H), 7.00 (m, 4H), 7.18 (m, 2H), 7.28 (m, 6H), 7.42 (m, 1H), 7.65 (d, J=8.7 Hz, 4H), 8.00 (d, J=8.7 Hz, 4H), 8.33 (s, 1H).

UV λₘₐₓ=315 nm εₘₐₓ=81000 M⁻¹ cm⁻¹ (ethanol)

### Example 8

### Synthesis of 2-(1-benzyl-1H-pyrrol-2-yl)-4,6-bis[4-(6,6-dimethyl-4-oxo-4,5,6,7-tetrahydroindol-1-yl)phenylamino]-1,3,5-triazine

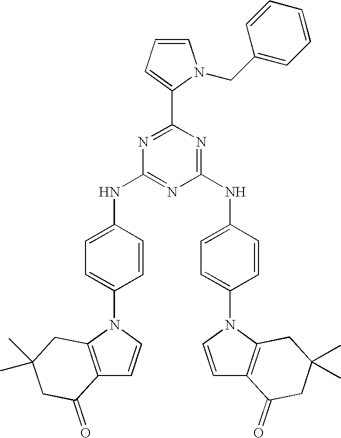

Yield: 44%.

¹H NMR (300 MHz, CDCl₃): δ 1.05 (s, 12H), 2.05 (s, 6H), 2.38 (m, 8H), 5.86 (s, 2H), 6.37 (s, 2H), 7.00 (m, 2H), 7.11 (d, J=8.4 Hz, 4H), 7.25 (m, 3H), 7.74 (d, J=8.4 Hz, 4H).

MS-EI (m/z): 769 (M+1).

### Example 9

### Synthesis of 2-(1-benzyl-1H-pyrrol-2-yl)-4,6-bis[4-((2-ethylhexyloxy)carbonyl)phenylamino]-1,3,5-triazine

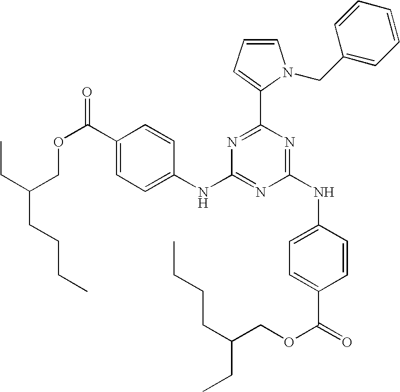

Yield: 44%.

¹H NMR (300 MHz, CDCl₃): δ 0.95 (m, 12H), 1.40 (m, 16H), 1.72 (m, 2H), 4.23 (m, 4H), 5.83 (s, 2H), 6.32 (s, 1H), 6.93 (m, 1H), 6.98 (d, J=7 Hz, 2H), 7.26 (m, 3H), 7.48 (m, 1H), 7.63 (d, J=8.4 Hz, 4H), 7.97 (d, J=8.4 Hz, 4H). MS-EI (m/z): 731 (M+1).

UV: λₘₐₓ=315 nm, εₘₐₓ=76000 M⁻¹ cm⁻¹ (CHCl₃/MeOH).

### Example 10

### Synthesis of 2-(1-benzyl-1H-pyrrol-2-yl)-4,6-bis[4-(H-imidazo[1,2-a]pyridin-2-yl)phenylamino]-1,3,5-triazine

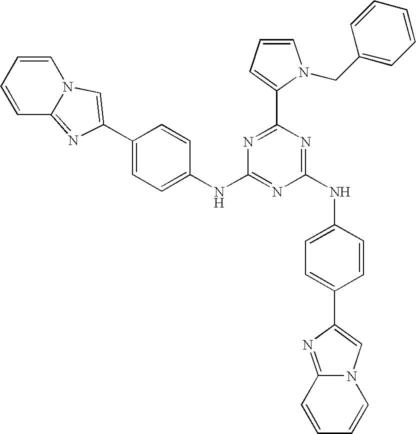

Yield: 14%.

S-EI (m/z): 727 (M+1).

UV: λₘₐₓ=338 nm, εₘₐₓ=55000 M⁻¹ cm⁻¹ (DMSO).

### Example 11

### Synthesis of 2-(1-benzyl-1H-pyrrol-2-yl)-4,6-bis[4-(1H-benzo[d]imidazol-2-yl)phenylamino]-1,3,5-triazine

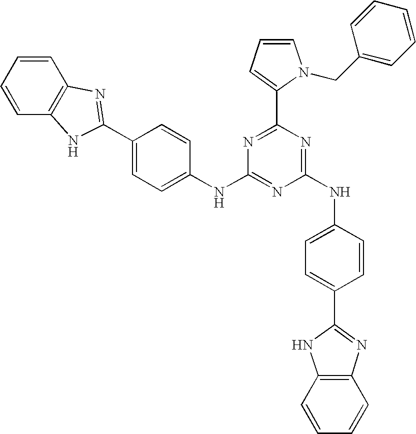

Yield: 65%.

¹H NMR (300 MHz, CDCl₃): δ 5.99 (s, 2H), 6.25 (m, 1H), 7.07 (m, 1H), 7.23 (m, 9H), 7.62 (m, 4H), 8.00 (m, 4H), 8.17 (m, 4H), 9.99 (s, 2H).

MS-EI (m/z): 651 (M+1).

UV: λₘₐₓ=327 nm, εₘₐₓ=100000 M¹ cm⁻¹ (CHCl₃/MeOH).

### Example 12

### Synthesis of 2-(1-benzyl-1H-pyrrol-2-yl)-4,6-bis[4-(1H-pyrazol-1-yl)phenylamino]-1,3,5-triazine

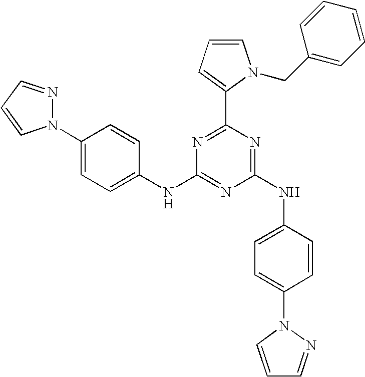

Yield: 33%.

¹H NMR (300 MHz, CDCl₃): δ 5.95 (s, 2H), 6.21 (m, 1H), 6.51 (m, 2H), 7.02 (m, 2H), 7.23 (m, 5H), 7.75 (m, 8H), 7.82 (m, 2H), 8.42 (m, 2H), 9.70 (s, 2H).

MS-EI (m/z): 551 (M+1).

UV: λₘₐₓ=307 nm, εₘₐₓ=67000 M⁻¹ cm⁻¹ (MeOH).

### Example 13

### Synthesis of 2-(1-benzyl-1H-pyrrol-2-yl)-4,6-bis[naphthalen-2-ylamino]-1,3,5-triazine

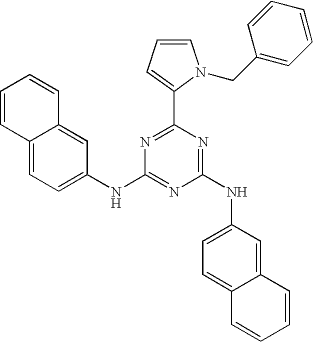

Yield: 65%.

¹H NMR (300 MHz, CDCl₃): δ 5.87 (s, 2H), 6.31 (m, 1H), 6.90 (m, 1H), 6.96 (m, 1H), 7.24 (m, 5H), 7.41 (m, 6H), 7.58 (m, 4H), 7.79 (m, 4H), 8.16 (s, 2H).

HPLC-MS/ES (m/z): 519 (M+1).

UV: λₘₐₓ=263 nm, εₘₐₓ=54000 M⁻¹ cm⁻¹, λₘₐₓ=308 nm, εₘₐₓ=52000 M⁻¹ cm⁻¹ (MeOH).

### Example 14

### Synthesis of 2-(1-benzyl-1H-pyrrol-2-yl)-4,6-bis[4-((E)-3-ethoxy-3-oxoprop-1-enyl)phenylamino]-1,3,5-triazine

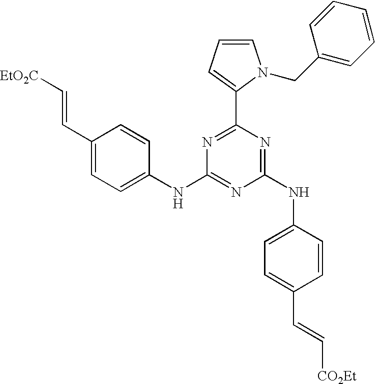

Yield: 16%.

¹H NMR (300 MHz, CDCl₃): δ 1.22 (m, 6H), 4.17 (m, 4H), 5.80 (m, 4H), 6.30 (m, 1H), 6.55 (m, 1H), 6.93 (m, 2H), 7.20 (m, 5H), 7.35 (m, 1H), 7.58 (m, 8H), 10.60 (s, 2H).

HPLC-MS/ES (m/z): 615 (M+1).

UV: λₘₐₓ=336 nm, εₘₐₓ=57000 M⁻¹ cm⁻¹ (MeOH).

### Example 15

### Synthesis of 2-(1-benzyl-1H-pyrrol-2-yl)-4,6-bis[4-(E)-styrylphenylamino]-1,3,5-triazine

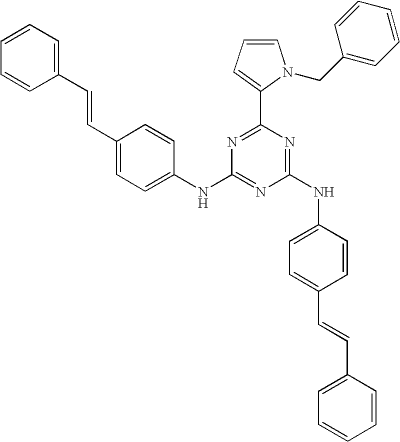

Yield: 50%.

¹H NMR (300 MHz, CDCl₃): δ 5.97 (m, 2H), 6.27 (m, 1H), 7.15 (m, 1H), 7.24 (m, 12H), 7.36 (m, 4H), 7.59 (m, 8H), 7.75 (m, 4H), 9.95 (s, 2H).

UV: λₘₐₓ=336 nm, εₘₐₓ=102000 M¹ cm⁻¹ (CHCl₃).

### Example 16

### Formulation in Oil

| | % w/w |
|---|---|
| Mineral Oil (Liquid Paraffin) | 59.85 |
| Arlamol HD (Uniqema) (Isohexadecane) | 16.00 |
| Arlamol S7 (Uniqema) (cyclomethicone, | 16.00 |
| PPG-15 estearil ether) | |
| ParsolMCX (DSM) (ethylhexyl methoxycinnamate) | 5.00 |
| Perfume | 0.15 |
| 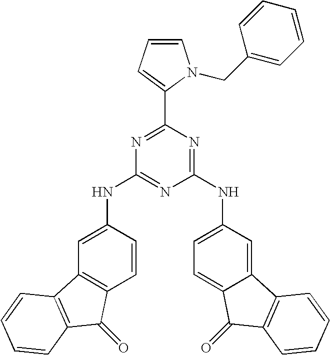 | 3.00 |

### Example 17

### Formulation in Form of Oil/Water Cream

| | % by weight |
|---|---|
| A) PEG-100 Stearate (Simulsol M59 (Seppic)) | 2.00 |
| Glyceryl Stearate (Cutina MS (Henkel)) | 1.00 |
| Cetearyl Alcohol (Lanette O (Henkel)) | 2.50 |
| Stearic Acid | 5.00 |
| Propyleneglycol | |
| Dicaprylate/dicaprate (Estol 1526 PDCC) | 7.50 |
| Triglyceride (Myritol 318 (Henkel) | |
| Caprylic/capric | 3.00 |
| Dimethicone (SF 18-350 (General Electric) | 0.50 |
| Tocopheryl acetate | 0.50 |
| 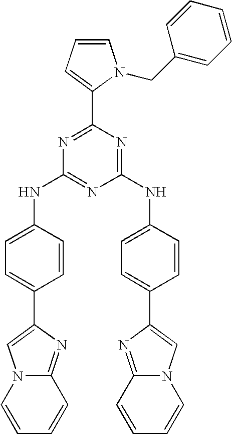 | 6.00 |
| B) Titanium Dioxide (y) | |
| Aluminium Hydroxide (y) | |
| Stearic Acid(MT-T100 TV (Tayca)) | 4.00 |
| Isohexadecane (Permethyl 101A (Presperse) | 5.00 |
| Cyclomethicone (SF 1204 (General Electric) | 2.50 |
| C) Water | up to 100 |
| Potassium Cethylphosphate (Amphisol K (Roche)) | 0.50 |
| D) PNC 30 (Sodium Acrylates/Crosslinked Polymer | 0.15 |
| Vinyl Isodecanoate) | |
| E) Butyleneglycol | 1.50 |
| Urea Imidazolidinyl | 0.30 |
| Methylparaben | 0.20 |
| Propylparaben | 0.10 |
| F. Perfume | 0.30 |

### Example 18

### Formulation in Gel Form

| | % by weight |
|---|---|
| A) Ethanol (95°) (CTFA: SD Alcohol) | 50.00 |
| Klucel HF (CTFA: hydroxypropylcellulose) | 2.00 |
| B) Ethanol (95°) (CTFA: SD Alcohol) | 27.50 |
| 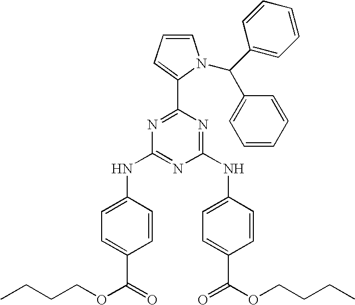 | 1.00 |
| PARSOL ® MCX (CTFA: octyl methoxycinammate) | 7.50 |
| Uvinul M-40 (CTFA: 3-benzophenone) | 4.00 |
| Finsolv TN (CTFA: C12-15 Alkyl Benzoate) | 5.00 |
| Fluied Silicone 556 (CTFA: Phenyl dimethicone) | 1.00 |
| C) Perfume, preservatives, deionised water | e.q.f. 100 |

### Example 19

### Formulation in Solid Stick Form

| | % by weight |
|---|---|
| 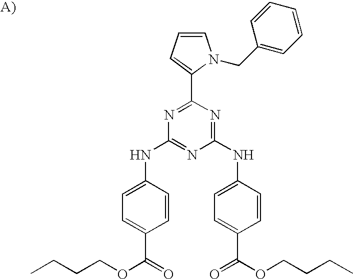 | 2.00 |
| PARSOL MCX (CTFA: Octyl Methoxycinnamate) | 7.50 |
| RICINOL (CTFA: Castor Oil) | 7.50 |
| Cutina HR (CTFA: Hydrogenated Castor Oil) | 7.50 |
| SATOL (CTFA: Oleyl Alcohol) purified and stabilised | 20.00 |
| Multiwax MH 180 (CTFA: Microcrystalline Wax) | 30.00 |
| Mineral Oil (30-40 ce) (CTFA) | 7.35 |
| Vaseline (CTFA: Petrolatum) | 8.53 |
| Silicone 200 Fluid (200 cs) (CTFA: Dimethicone) | 3.50 |
| Butyl hydroxytoluene (CTFA: BHT) | 0.02 |
| Betacarotene (sol'n at 1%) | 0.30 |
| B) d-PANTENOL (CTFA: Panthenol) | 0.80 |
| Propyleneglycol (CTFA: Propylenglycol) | 3.00 |
| C) Perfume, preservatives, vaseline | e.q.f. 100 |

### Example 20

### Formulation in Fluid Form

| | % |
|---|---|
| | by weight |
| 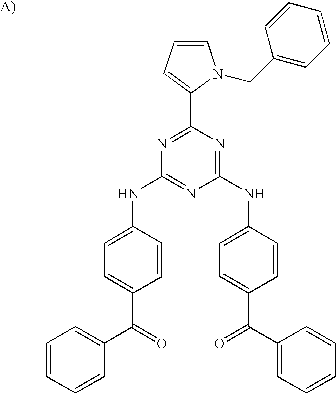 | 1.00 |
| PARSOL ® MCX (CTFA: Octyl methoxycinnamate) | 6.00 |
| Uvinul M-40 (CTFA: 3-Benzophenone) | 0.50 |
| Silicone 344 fluid (CTFA: Cyclomethicone) | 45.00 |
| Silicone Q2-1401 (CTFA: Cyclomethicone (y) Dimethicone) | 20.00 |
| Finsolv TN (CTFA: C₁₂-C₁₅ Alcohol Benzoates) | 10.00 |
| Crodamol DA (CTFA: Diisopropyl Adipate) | 15.50 |
| B) Perfume, Finsolv TN | e.q.f. 100 |

### Example 21

### Formulation in Water/Oil Emulsion Form

| | % by weight |
|---|---|
| A) Arlacel P135 (PEG-30 Dipolyhydroxyestearate) | 4.00 |
| Arlamol HD (Isohexadecano) | 6.00 |
| Oil Mineral (Liquid Paraffin) | 3.00 |
| Tioveil 50 FCM (Titanium Dioxide, C₁₂-C₁₅ alkyl benzoate, Cyclomethicone, polyhydroxystearic, | 12.00 |
| aluminium stearate, alumina) | |
| 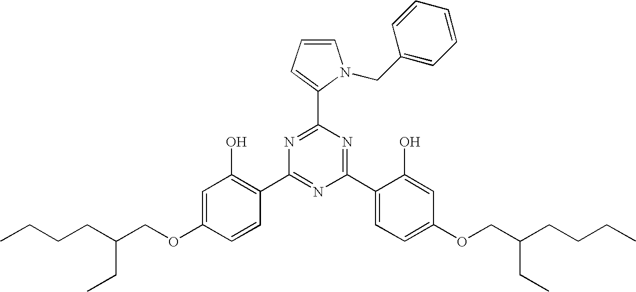 | 6.00 |
| B) Water up to | 100.00 |
| Pricerine 9091 (Glycerine) | 3.00 |
| MgSO₄•7H₂O (Magnesium sulphate) | 0.70 |
| Alpantha (Panthenol, Allantoin) | 0.30 |
| C) Kemaben 2 (Propyleneglycol, diazolidinil urea, methylparaben, Propylparaben) | 1.00 |
| D) Perfume | e.q. |

### Example 22

### Formulation as Cream for the Prevention and/or as Coadjuvant in Treatment of Melasma

| | % by weight |
|---|---|
| 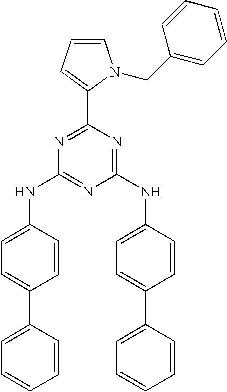 | 10.00 |
| Parsol MCX (ethylhexyl methoxycinnamate) | 6.00 |
| Hydroquinone | 4.00 |
| Uvinul M-40 (benzophenone-3) | 2.00 |
| Parsol 1789 (Butyl methoxydibenzoylmethane) | 1.50 |
| Evanescent foundation cream | e.q.f. 100 |

1. A pyrrolyltriazine derivative of general formula (I): 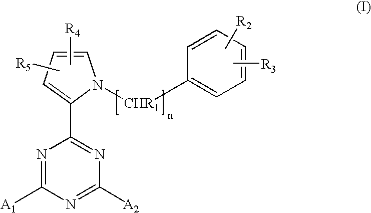
wherein
n=1;
R₁ represents an atom of hydrogen; a linear or branched alkyl radical having from 1 to 3 atoms, optionally substituted; or a substituted R₂′, R₃′ phenyl radical;
R₂, R₂′, R₃ and R₃′ are the same as or different from each other and represent an atom of hydrogen; an optionally substituted linear or branched alkyl radical having from 1 to 3 carbon atoms; an alkoxy radical having from 1 to 3 carbon atoms; an aryl radical; halogen or hydroxyl radical; or else
R₂ and R₃ are condensates that form with the phenyl a naphthalene ring, optionally substituted;
R₄ and R₅ are the same as or different from each other and represent an atom of hydrogen; an optionally substituted linear or branched alkyl radical having from 1 to 4 carbon atoms; or an optionally substituted aryl radical;
A₁ is a radical of general formula (II), (III) or (IV) 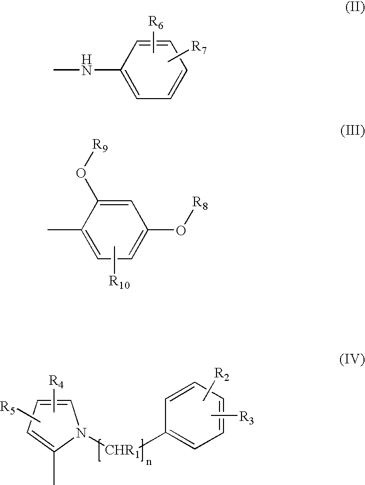
A₂ is a radical of general formula (II) or (V) 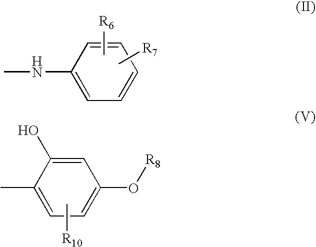
R₆ represents an atom of hydrogen; an optionally substituted, linear or branched chain, saturated or unsaturated alkyl radical that contains from 1 to 6 carbon atoms; or a hydroxyl radical;
R₇ represents an atom of hydrogen; an optionally substituted aryl radical; an optionally substituted saturated, unsaturated or aromatic heterocyclic radical having from 5 to 10 atoms that can contain 1, 2 or 3 heteroatoms selected from O, N and S; a —COOR₁₁ radical; a —CONR₁₂R₁₃ radical; an alkoxy radical having from 1 to 18 carbon atoms optionally substituted; an optionally substituted aryloxy radical; an optionally substituted —COR₁₄ radical; a C₃-C₆ cycloalkyl radical; a linear or branched chain, saturated or unsaturated alkyl radical that contains from 1 to 18 carbon atoms, optionally substituted with at least one hydroxyl radical, an SO₃M or —N(R₁₅)₃⁺ group or else a group of general formula (VI) 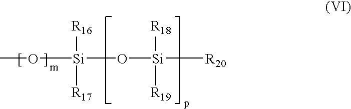
in which
m=0 or 1;
p=0, 1, 2, 3 or 4;
R₁₆, R₁₇, R₁₈, R₁₉ and R₂₀ are the same as or different from each other and represent an optionally substituted alkyl radical having from 1 to 6 carbon atoms; an alkoxy radical having from 1 to 6 carbon atoms, an optionally substituted aryl radical or an —OSi(R₂₁)₃ radical;
R₂₁ represents an alkyl radical of 1 to 6 carbon atoms, an alkoxy radical of 1 to 6 atoms of carbon or an optionally substituted aryl radical;
R₁₁, R₁₂ and R₁₃ are the same as or different from each other and represent an atom of hydrogen; an alkyl radical, linear or branched chain having from 1 to 18 carbon atoms optionally substituted; a C₃-C₆ radical cycloalkyl, or else
R₁₂ and R₁₃ form together with the nitrogen atom a saturated heterocylic compound having from 5 to 7 carbon atoms that can contain 1, 2 or 3 heteroatoms selected from O, N and S;
R₁₄ is an optionally substituted alkyl radical or an optionally substituted aryl radical;
R₁₅ is an optionally substituted alkyl radical;
M is H, Na or K;
R₆ and R₇ or else, R₆ and R₁₄ are condensates forming with the phenyl a polycyclic system having from 9 to 15 atoms, optionally substituted;
R₈ and R₉ can be the same as or different from each other and represent an atom of hydrogen; an optionally substituted acyl radical having from 1 to 18 carbon atoms; a linear or branched, saturated or unsaturated alkyl radical that contains from 1 to 18 carbon atoms, optionally substituted with at least one hydroxyl radical, an SO₃M or —N(R₁₅)₃⁺ group or else a group of general formula (VI), as defined above;
R₁₀ represents an atom of hydrogen or a SO₃M radical, with M being as defined above.

2. A derivative as defined in claim 1, in which A₁ and A₂ are the same and represent a radical of general formula (II) or one of general formula (V): 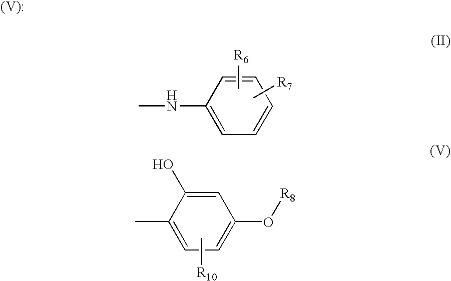
with R₆, R₇, R₈ and R₁₀ being as defined in claim 1.

3. A derivative according to claim 1, which has general formula (IA): 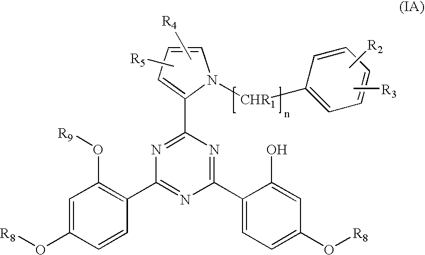
with R₁, R₂, R₃, R₄, R₅, R₈, R₉ and n being as defined in claim 1.

4. A derivative according to claim 1, which has general formula (IB): 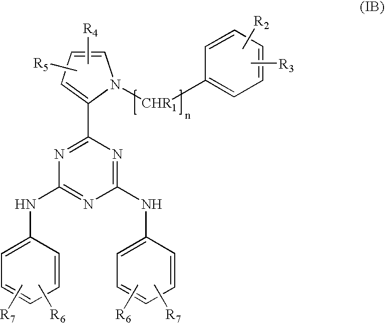
with R₁, R₂, R₃, R₄, R₅, R₆, R₇ and n being as defined in claim 1.

5. A derivative according to claim 1, in which R₁ represents hydrogen, alkyl, phenyl or phenylalkyl, optionally substituted in at least one position by a phenyl, chloro, bromo, fluoro, alkoxy or alkyl group.

6. A derivative according to claim 1, in which R₂ and R₂′ represent hydrogen, phenyl, methyl or ethyl.

7. A derivative according to claim 1, in which R₃ and R₃′ represent hydrogen, phenyl, methyl or ethyl.

8. A derivative according to claim 1, in which R₂ and R₃ form a naphthalene ring.

9. A derivative according to claim 1, in which R₄ and R₅ are the same as or different from each other and represent hydrogen, methyl or phenyl.

10. A derivative according to claim 1, in which R₆ represents hydrogen, hydroxyl, methyl or ethyl.

11. A derivative according to claim 1, in which R₇ represents hydrogen, hydroxyl, methyl, ethyl, tert-butyl, benzyl, cyclohexyl, methoxyphenyl, biphenyl, COOR₁₁, CONR₁₂R₁₃ or 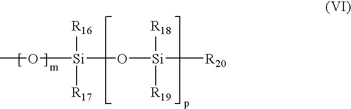
with R₁₁, R₁₂, R₁₃, R₁₆, R₁₇, R₁₈, R₁₉, R₂₀, m and p being as defined in claim 1.

12. A derivative according to claim 1, in which R₈ represents ethylhexyl or a linear or branched chain, saturated or unsaturated alkyl radical that contains from 1 to 18 carbon atoms, optionally substituted with at least one —SO₃M or —N(R₁₅)₃⁺ group; and R₉ is H.

13. A derivative according to claim 1, in which R₁₁ represents hydrogen, methyl, ethyl, propyl, butyl, tert-butyl, pentyl, hexyl or 2-ethylhexyl.

14. A derivative according to claim 1, in which R₁₂ represents hydrogen, methyl, ethyl, propyl, butyl, tert-butyl, pentyl, hexyl or 2-ethylhexyl.

15. A derivative according to claim 1, in which R₁₃ represents hydrogen, methyl, ethyl, propyl, butyl, tert-butyl, pentyl, hexyl or 2-ethylhexyl.

16. A derivative according to claim 1, in which R₁₄ represents methyl, ethyl, propyl, butyl t-butyl or phenyl.

17. A derivative according to claim 1, in which R₁₆ to R₂₀ represent methyl, ethyl, methoxy, ethoxy or phenyl.

18. A derivative according to claim 1, in which R₂₁ represents methyl, ethyl, methoxy, ethoxy or phenyl.

19. A derivative according to claim 1, selected from the group consisting of:
2-(1-benzyl-1H-pyrrol-2-yl)-4,6-bis(2,4-dihydroxyphenyl)-1,3,5-triazine;
2-(1-benzyl-1H-pyrrol-2-yl)-4,6-bis[4-(2-ethylhexyloxy)-2-hydroxyphenyl]-1,3,5-triazine;
2-(1-benzyl-1H-pyrrol-2-yl)-4,6-bis[4-(butoxycarbonyl)phenylamino]-1,3,5-triazine;
2-(1-benzyl-1H-pyrrol-2-yl)-4,6-bis(biphenyl-4-ylamino)-1,3,5-triazine;
2-(1-benzyl-1H-pyrrol-2-yl)-4,6-bis(4-benzoylphenylamino)-1,3,5-triazine;
2-(1-benzyl-1H-pyrrol-2-yl)-4,6-bis(9-oxo-9H-fluoren-3-ylamino)-1,3,5-triazine;
2-(1-benzyl-1H-pyrrol-2-yl)-4,6-bis[4-(imidazo[1,2-a]pyrridin-2-yl)phenylamino]-1,3,5-triazine;
2-(1-benzydryl-1H-pyrrol-2-yl)-4,6-bis[4-(butoxycarbonyl)phenylamino]-1,3,5-triazine.

20. (canceled)

21. A method for preparing a pyrrolyltriazine derivative of general formula (I) according to claim 1, in which
A₁ is a radical of general formula (III) 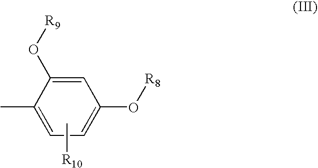
and A₂ is a radical of general formula (V) 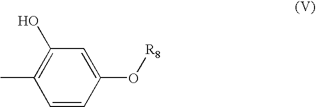
with R₁₀=H,
which includes alkylation of a compound of general formula (IX) with a compound of general formula (X) 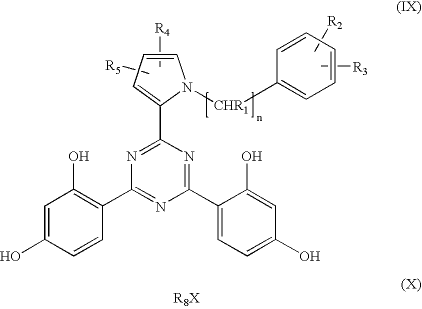
with R₁-R₅, R₈, and n being as defined in claim 1 and X being a leaving group such as chloro, bromo, tosyl or mesyl, in a basic medium with a polar solvent, which base is selected from sodium hydroxide, potassium hydroxide, cesium carbonate, potassium carbonate, sodium tert-butoxide and potassium tert-butoxide and which polar solvent is selected from 2-methoxyethanol, 2-ethoxyethanol, dimethylformamide or ethanol.

22. (canceled)

23. (canceled)

24. (canceled)

25. (canceled)

26. A pyrrolyltriazine derivatives according to claim 1 for use as a UV radiation absorbing agent.

27. A cosmetic dermatological or pharmaceutical formulation that comprises one or more derivatives according to claim 1 and at least one cosmetically, dermatologically or pharmaceutically acceptable carrier or excipient.

28. (canceled)

29. (canceled)

30. A formulation according to claim 27, which also comprises at least one compound selected from the group consisting of an organic, micronised organic or inorganic filter against solar radiation, and one active substance.

31. (canceled)

32. A method of use of a pyrrolyltriazine derivative according to claim 1 as a UV radiation filtering agent in a cosmetic, dermatological and/or pharmaceutical composition , the method comprising administering to a subject a cosmetically, dermatologically and/or pharmaceutically effective amount of a pyrrolyltriazine derivative according to claim 1 together with cosmetically, dermatologically and or pharmaceutically acceptable carriers or excipients.

33. A method of use of a pyrrolyltriazine derivatives according to claim 1 for the protection, prophylaxis and/or coadjuvant treatment of healthy or diseased skin, lips and/or related tissues of a mammal against ultraviolet radiation, the method comprising administering to a subject a cosmetically, dermatologically and/or pharmaceutically effective amount of a pyrrolyltriazine derivative according to claim 1 together with cosmetically, dermatologically and/or pharmaceutically acceptable carriers or excipients.

34. (canceled)

35. (canceled)

36. A method of use of a derivative according to claim 1 as a photostabiliser of synthetic polymers or as an ultraviolet radiation filtering agent in textile fibres, the method comprising administering to a subject a photostabilizing and/or filtering effective amount of a pyrrolyltriazine derivative according to claim 1.

37. (canceled)
